(19) United States
(12) Patent Application Publication
(10) Pub. No.: US 2021/0196435 A1
(43) Pub. Date: Jul. 1, 2021

(54) Process for Producing a Molded Product

(71) Applicants: Vita Zahnfabrick H. Rauter GmbH & Co. KG, Bad Säckingen (DE); Imes-Icore GmbH, Eiterfeld (DE); Technische Universität Darmstadt, Darmstadt (DE); Form for Function Fräszentrum GmbH, Darmstadt (DE)

(72) Inventors: Thomas Jobst, Darmstadt (DE); Christof Ellerbrock, Ingelheim (DE); Armin Kirsten, Bad Säckingen (DE); Christoph Stark, Fulda (DE); Sebastian Ullrich, Hünfeld (DE); Christian Steinmetz, Dreieich (DE); Reinhard Heister, Rodgau (DE); Vitali Dejkun, Darmstadt (DE); Stefan Mischliwski, Worms (DE)

(21) Appl. No.: 16/756,884

(22) PCT Filed: Nov. 8, 2018
(86) PCT No.: PCT/EP2018/080649

§ 371 Date: Apr. 17, 2020

(30) Foreign Application Priority Data
Nov. 10, 2017 (EP) 17201063.9

(51) Int. Cl. A61C 13/00; A61C 13/083

(52) U.S. Cl. CPC A61C 13/0004; A61C 13/0018; A61C 13/0835; A61C 13/0022

(57) ABSTRACT

The present invention relates to a mechanized and automated process for producing a molded product, and to a molded product produced by said process.

The present invention relates to a mechanized process for producing a molded product, and to a molded product produced by said process.

The precisely tailored and accurate manufacturing of a unique individual or irregular molded product is usually associated with a high economic and technical expenditure. Although the production is computer-supported in many cases, for example, by digitalizing the prototype and manufacturing through CAD/CAM modules, a manual human post-processing and adaptation cannot be dispensed with in most cases in the final processing. This is the case, in particular, if other properties in addition to precise tailoring and accuracy, such as the appearance of the molded product, are of importance to the respective application. There may be mentioned, in particular, medical and cosmetic uses, for example, in the field of orthopedic and dental restorations. Even in fields where the optical properties are not paramount, such as in the automobile branch or mechanical engineering and mold construction, the surface and functional properties of a molded product represent an important means in the adaptation of the physical properties to the respective requirements. Thus, for example, the resistance of a molded product towards mechanical, physical and chemical loads can be influenced by a suitable design of the surface and the material composition.

The production of a corresponding molded product is further complicated by the fact that in many cases the optimum molded product may consist of different materials and material layers, and the production must be effected in several process steps. Thus, in particular, the production of multilayered molded products, for example, by veneering in the dental field, is very work-intensive, since the individual layers must be applied and processed individually and manually in each case.

In the prior art, a number of processes are known that describe the production of a molded product by machine-supported methods.

DE 10 2010 037 160 describes a method for the production of dentures, in which for the production of veneers on a support frame, several layers of at least one material mixture are applied to a spatially curved exterior surface of a support frame, in an automated, particularly a computer-controlled fashion according to a digital model of the dentures, with the layers of the material mixture being applied as layers arranged in a spatially curved manner. The method is characterized in that several layers of the material mixture are applied directly following each other.

DE 199 22 870 discloses a method for the automated, individually adapted creation of the color, translucency, brightness and fluorescence of dental restorations, wherein the basic steps of the method reside in data acquisition, generation of a CAD data set for describing the shape, and CAD/CAM data sets for applying the layers, followed by fully automated testing of the result, comparison with the specification, one or more ablations of parts of the layer that may be required, correction of the data input, and renewed application.

DE 10 2009 011 175 relates to a method for the automatic dental ceramic veneering of frameworks of dental restorations using a manufacturing device comprising at least one holding and positioning unit, at least one coating unit comprising at least one coating nozzle, a control unit and, preferably a furnace chamber, wherein a CAD/CAM data record is used for the coating application of a veneering, and the positional change of the framework relative to the nozzle during the application process is merely effected by moving the holding and positioning unit relative to the nozzle. Further, the manufacturing device is characterized in that the holding and positioning unit can be rotated around five axes or more, and the nozzle has a stationary design.

US 2004/245663 A1 discloses a method for producing molded products in which tapes based on silicone polymers are applied to ceramic frameworks.

The methods known in the prior art have the disadvantage that the final processing must be effected manually in most cases, or by having to perform complicated measurements for checking the result. Thus, the additive and subtractive process steps must usually be performed separately, either in different machines or in part manually.

Another drawback of the methods described in the prior art is that the additive process steps are performed by using a laser in most cases. This results in a thermal load that may have negative effects on the substrate.

Further, the methods described in the prior art do not allow several materials or different colors to be applied successively by automated application.

US 2016/0129528 describes, inter alia, a method for applying a coating to a component in which the coating is provided in the form of a film and molten onto the component by using a laser.

US 2017/0057011 and US 2017/0008127 relate to a printing head for applying a material to a component. The component is clamped into a first holder, and the printing head is clamped into a second holder. Part of the substrate is bombarded with the material to be applied and heated during such application.

Therefore, there is still a need for a fully automated method that allows for the production of a multilayered molded product with a high dimensional accuracy and different individually adapted layer and material properties to obtain optimum properties, especially in terms of function and appearance, of the molded product, in which the work-intensive manual post-processing can be dispensed with. Further, there is a need for a fully automated method in which the thermal load on the substrate is preferably kept low.

Therefore, it is the object of the present invention to provide a process that allows for the fully automated production of a dimensionally accurate molded product with different materials and material properties, in which the functionality and appearance of the individual layers are paramount, which can be specifically adapted depending on the intended application.

Surprisingly, it has been found that this object can be achieved by a process that is based on a combination of subtractive and additive process steps, in which both the subtractive processing and the additive processing are performed by a mechanized and automated method.

Therefore, the present invention firstly relates to a process for producing a molded product, comprising the following steps:
a) providing a blank;
b) machine-processing the blank using subtractive methods to obtain a framework structure;
c) machine-applying a coating to the framework structure obtained in step b) to obtain a raw molded product;
d) curing the coating;
e) machine-processing the coating using subtractive methods to obtain the desired molded product, wherein the application of the coatings, in particular, is effected using a device having an automated cartridge changer.

A highly aesthetic crown restoration in the front tooth region requires an optimum imitation of the natural tooth appearance and function. In order to achieve this object, the complicated manual work of an experienced dental technician is mandatory today. In part, the reconstruction of a single tooth may involve several hours of manual work. The process according to the invention should enable not only an efficiency enhancement of the processes in a dental laboratory by the machine implementation of the complicated process, but also an enhancement of the reproducibility, process safety and functionality of the molded product.

Depending on the application, it may be advantageous to apply more than one coating to the framework structure. Therefore, an embodiment is preferred in which the process according to the invention includes the following steps:
a) providing a blank;
b) machine-processing the blank using subtractive methods to obtain a framework structure;
c) machine-applying a coating to the framework structure obtained in step b) to obtain a raw molded product;
d) curing the coating;
e) machine-processing the coating using subtractive methods;
f) machine-applying a further coating, which preferably has a different coating composition in terms of color, property or functionality from that in step c), to obtain an extended raw molded product;
g) curing the further coating;
h) machine-processing the further coating using subtractive methods;
i) repeating steps f) to h) to obtain the desired molded product.

Conventional methods have the disadvantage that the application of the coating to the blank, in particular, must be effected manually in order to achieve the desired and demanded quality, especially in optical terms. This way of manufacturing is additionally complicated by the fact that in most cases the applying of a single coating is not sufficient to achieve the desired result, wherein each coating must be applied and brought into the desired shape by tedious manual work. This manual processing, which requires a high skill and a qualified worker who is able to perform such a demanding task, is usually followed by further processing steps, such as polishing, which must also be performed manually.

The process according to the invention offers the advantage that all steps are performed in a mechanized method, wherein all process steps are preferably performed in the same machine or by the same machine. Thus, the processing of the blank or the coating by subtractive methods is also performed in a mechanized method, as is the application of the coating to the blank. A “mechanized method” within the meaning of the present invention means working steps that are performed by a machine without direct human intervention.

In a preferred embodiment of the process according to the invention, the process steps are performed in the stated order.

The process according to the invention is characterized in that molded products true to color, function and shape can be produced without manual post-processing being necessary. Therefore, an embodiment in which the process is automated is preferred. “Automated” within the meaning of the present invention means the performance of the individual process steps without involvement of human resources. However, it is not excluded that quality control, especially the final one, and further working steps, such as the detaching of the blank from the holder, a final polishing or painting, may be done manually. Also, human intervention to take control remains reserved. It is not against the automated performance of the process according to the invention that certain data, for example, relating to the performing of the subtractive method or the application of the coatings, is provided by a human employee. The automated application is preferably effected by using a device that includes means for receiving and delivering different materials. These means are preferably cartridges that can receive and deliver the material employed, in which the delivering can be done by using a nozzle, for example. More preferably, the cartridges are arranged in a way that allows the cartridges to be exchanged automatically.

Depending on the desired application and function of the molded product, it may make sense to apply one or more further coatings having different material properties and colors, in addition to the coating already applied. Therefore, an embodiment is preferred in which, in addition to said coating, one or more further coatings are applied thereto. This is preferably performed by analogy with the application of the first coating, especially by repeating steps c) to e) of the process according to the invention, to obtain the desired molded product.

The process according to the invention is suitable, in particular, for the production of molded products on whose optical and functional properties high demands are placed. Thus, mainly in the field of dental restorations, there is a challenge that the appearance and the property of a natural tooth are imitated as naturally as possible. In particular, it is to be considered that each tooth has an individual color gradient, which is coined, for example, by the eating and living habits of its owner. Even the individual teeth of a person have different appearances, so that the difficulty is to fit the dental restoration into the existing tooth and color scheme to form as natural as possible an appearance. In conventional methods, such color gradients are usually achieved by applying different colored coatings manually to a fundamental framework, wherein each coating must be accordingly adapted to the desired shape. Thus, whether a unitary appearance can be achieved primarily depends on the experience, dexterity and color perception of the person who produces the dental restoration. The process according to the invention here offers optimization because all steps, including the application of the coatings and the processing thereof, are effected by a mechanized method. Therefore, an embodiment is preferred in which the molded product is a dental restoration. Said dental restoration may be inlays, onlays, bridges, crowns or implants, for example.

Especially in the production of dental restorations, the advantages of the process according to the invention are revealed. Thus, the process according to the invention allows for an imitation of the human tooth that is close to nature, not only with respect to the optical properties, but also with respect to functionality and mechanical properties.

However, the process according to the invention is not limited to the production of dental restorations. Rather, it may also be used for the production of molded products in other technical fields, for example, in the fields of plant and mechanical engineering and mold construction, electrical engineering, production technology, orthopedic/medical technology, or automobile construction.

In the following, the individual process steps are explained in more detail.

### Process Step a)

Step a) of the process according to the invention includes the providing of a blank. The blank is not subject to any limitation either in shape of in material terms. Preferably, the blank comprises one or more materials selected from the group consisting of metallic materials, polymer-based materials and ceramic materials, as well as mixtures thereof. The material and composition of the blank can be selected as a function of the respective application.

The process according to the invention is particularly suitable for the production of dental restorations. Therefore, materials are preferred that are employed for the production of dental restorations. Therefore, in a preferred embodiment, the blank comprises a composite material. A “composite” within the meaning of the present invention means a composite material that is constituted by two or more materials bonded together.

In a particularly preferred embodiment, the composite material consists of an organic plastic matrix admixed with inorganic packings. Said packings may include, for example, glasses and glass ceramics, silicates and silicon dioxide.

In an alternatively preferred embodiment, the blank is a ceramic, especially a glass ceramic or a zirconia ceramic. In a particularly preferred embodiment, the blank is a hybrid ceramic. A “hybrid ceramic” within the meaning of the present invention means a ceramic, for example a glass ceramic or a zirconia ceramic, admixed with a polymer-based filler.

Depending on the field of application of the molded product, it may be advantageous for the blank to include a metallic material. Therefore, an embodiment is preferred in which the blank is an alloy.

### Process Step b)

According to step b) of the process according to the invention, the provided blank is machine-processed using subtractive methods to obtain a framework structure. The subtractive methods are preferably selected from the group consisting of milling, grinding, laser ablation, and water jet cutting. Preferably, the subtractive method is a CAD/CAM method. In this way, it is ensured that a high dimensional accuracy is achieved. Thus, for example, a computer-based data set that describes the framework structure can be generated. This data set can then be used as a basis for the machine-processing of the blank.

In a preferred embodiment, the framework structure obtained in step b) of the process according to the invention is a framework structure in the form of an anatomically reduced single crown construction, or of an anatomically reduced multiple-unit bridge construction. The anatomic reduction of the outside contour preferably represents an imitation of the interior dentin form of a natural tooth.

### Process Step c)

The further processing of the framework structure obtained in step b) is performed by machine-applying a coating to obtain a raw molded product, as described under step c) of the process according to the invention.

The application of the coating to the framework structure can be effected by using any machine technologies. In a preferred embodiment, the application of the coating is effected by additive methods. Thus, in a particularly preferred embodiment, the application of the coating may be effected, for example, by extrusion, spraying, vapor deposition, deposition, infiltration, or immersion coating. In order to achieve an optimum result, several technologies may also be combined. Further, the application of the coating may also be effected with computer support using a CAD/CAM method in order to ensure an application that is as accurate and loss-free as possible.

In an embodiment of the present invention, the application of the coating is not effected by using a tape.

The coating material can be selected arbitrarily taking into consideration the respective application of the molded product and its compatibility with the material of the framework structure. In a preferred embodiment, the coating includes one or more materials selected from the group consisting of metallic materials, polymer-based materials, and ceramic materials, as well as mixtures thereof.

Since the process according to the invention is suitable for the production of dental restorations, in particular, coating materials are preferred that are compatible with the requirements on this technical field, especially in view of the optical and mechanical properties. Further, the material should be safe to health. Therefore, the coating material is preferably a composite material, a glass ceramic, a zirconia ceramic, or an alloy. Combinations of such material types may also be employed.

In a particularly preferred embodiment, the coating material is a polymer-based material. Suitable materials may include, for example, polymer-based plastics from the group of methacrylates (e.g., PMMA, bis-GMA, UDMA, TEGDMA) or composites including a plastic material from the above group and additionally inorganic fillers (e.g., glasses, ceramics, glass ceramics). The coating is preferably effected in a partially polymerized or unpolymerized state of the polymer fraction.

In a particularly preferred embodiment, the coating material is an inorganic material. Particularly preferred materials include silicate glasses or glass ceramics based on feldspar, lithium silicate, or leucite. In this case, the coating is preferably effected in a dispersed state.

The coating material may also be used to determine the physical properties of the subsequent molded product. The physical properties may include both the optical properties and mechanical properties, such as stiffness, density, strength or hardness. In particular, the process according to the invention here too allows for the production of a molded product whose different layers have different properties.

Thus, for example, a particular appearance of the molded product can be achieved by admixing the coating material with particular additives. Accordingly, an embodiment is preferred in which the coating material includes further additives. The additives may be, for example, coloring substances, such as coloring oxides, pigments or organic colorants. For example, the strength or the translucency of the molded product can be determined by adding suitable substances. Exemplary substances may include, in particular, colorants and glass-coloring oxides, wherein the selection of the added substance is not limited to those, but may be selected according to the respective individual demands required from the molded product.

Like the materials of the blank and of the coating are selected as a function of the requirements demanded from the subsequent molded product, the combination of materials of the blank and of the coating may also be selected in accordance with such requirements. In a preferred embodiment, the blank and the coating may be the same materials. In an alternatively preferred embodiment, the blank and the coating are different materials.

The process according to the invention is suitable for combining different materials, in particular. Preferably, the material and the coating material are different from one another, there being no limitation of the possible combinations.

In a preferred embodiment, the material of the blank is yttrium-stabilized zirconia to which a glass ceramic based on feldspar or leucite is applied as a coating.

In an alternatively preferred embodiment, a glass ceramic based on feldspar or leucite is applied as a coating to a dental alloy as a blank material.

A “dental alloy” within the meaning of the present invention is a generic term for corrosion-resistant, non-discoloring, abrasion-resistant alloys compatible with the dental and oral tissues for tooth preservation and tooth replacement in the form of crowns, bridges, pivot teeth, implants and prostheses. Such alloys may be on the basis of precious metals or base metals. Suitable materials and combinations of materials are known to the skilled person.

It is further alternatively preferred to use an yttrium-stabilized zirconia as a blank material, and a polymer-based composite material for the coating.

Alternatively, a dental alloy as the blank material is preferably coated with a polymer-based composite material.

Alternatively, in a preferred embodiment, a high-performance polymer from the group of polyaryletherketones (PAEK) (e.g., PEKK, PEEK) can be used as a blank material, and a polymer-based composite material for the coating.

In an also preferred embodiment, a hybrid ceramic is employed as a blank material, and a polymer-based composite material for the coating.

Further preferred is an embodiment in which a polymer-based composite material is used as a blank material, and a polymer-based composite material for the coating.

In a preferred embodiment of the invention, the material for the coating contains essentially no silicone polymers. In particular, preferably a proportion of less than 10% by weight, more preferably less than 5% by weight, especially less than 0.5% by weight, or 0% by weight of silicone polymer are present in the coating.

According to a preferred embodiment of the process according to the invention, more than one coating can be applied to the framework structure. In a preferred embodiment, the material of the first coating and of the further coating is the same material, while different materials are used in an alternatively preferred embodiment. For example, different coatings with different colors can be applied in order to thereby achieve a particular color gradient, or a particular translucency of the molded product. But also, in view of the mechanical strength, the required material properties or the machine processability, it may be of advantage to apply more than one coating having different layer properties to the framework structure.

In a preferred embodiment, the application of the coating is effected without using a laser for melting it on the surface. In this way, a thermal load on the material and on the framework structure, which would be disadvantageous, can be avoided.

### Process Step d)

The coating is cured after the application. The appropriate method depends on the material of the coating and on the degree of hardness to be attained. If different coatings of different materials are employed, it may be appropriate to apply or to combine different methods within one process run.

In a preferred embodiment, the curing of the coating is effected by polymerization, sintering, drying, cooling, pressure or irradiation. Also, different curing methods may be combined.

### Process Step e)

In a further step of the process according to the invention, the coating applied is machine-processed by subtractive methods after the curing to obtain the desired molded product. In this step, shape corrections may be performed, surfaces optimized, or excess coating material removed, for example. In a preferred embodiment, one or more further coatings may be machine-applied to the coating, as described in process steps d) to e). The further coatings or coatings are machine-applied to the previous coatings or coatings, and then cured. After the curing, the applied coating is machine-processed by subtractive methods, for example, in order to achieve a particular surface structure or to remove excess coating material. Further, this step can be used to perform optical improvements, for example, by polishing.

Depending on the combination of different materials, further surface treatment steps may be necessary to prepare a safe material composite. These surface treatment steps may be, for example, blasting with blasting means (corundum, glass beads, glass-coated corundum), or etching by means of acid (e.g., hydrofluoric acid, phosphoric acid). It is alternatively preferred that the surface treatment includes the application of an adhesion promoter, which may contain one or more compound types from the group of silane, phosphate ester, phosphonic acid and/or sulfur compound, respectively with a free-radically polymerizable group (e.g., methacryloyloxypropyltrimethoxysilane, methacryloyloxydecyl dihydrogenphosphate, vinylbenzylpropylamino-triazine-dithione).

Further preferably, the surface treatment may be effected by partially dissolving a superficial layer by means of a solvent (e.g., methyl methacrylate), or grinding over by using diamond-tipped grinding means.

### Additional Process Steps

The process according to the invention may include further process steps depending on the intended field of application of the molded product or as a function of the materials employed. Thus, it may be of advantage to effect a thermal treatment of the framework structure obtained after the machine-processing of the blank by means of subtractive methods. Therefore, an embodiment is preferred in which step b) of the process according to the invention is followed by a thermal treatment of the framework structure. Such a thermal treatment is advantageous, in particular, if the blank is made of a material that, during the subtractive processing, is not yet in the state having the material properties corresponding to its intended use. The thermal treatment may preferably pursuit the object of drying, debinding, sintering, a crystallization, a polymerization, or one or more combinations of these processes. Typical temperatures of such processes are known to the skilled person from the literature and can be selected as a function of the purpose of the thermal treatment and of the affected material. Thus, the temperatures at which debinding is performed, for example, usually range from 500° C. to 700° C., while the sintering of zirconia is performed at from 1000° C. to 1300° C., and the sintering of feldspar ceramics is done from 500° C. to 900° C.

The process according to the invention is suitable, in particular, for the production of molded products that are individually adapted to the respective requirements of their field of application. Therefore, the present invention further relates to a molded product obtainable by the process according to the invention. More preferably, the molded product is a dental restoration.

The present invention further relates to the use of a blank comprising one or more materials selected from the group consisting of ceramic materials, polymer-based materials, metallic materials, and mixtures thereof, in a process according to the present invention.

The present invention further relates to the use of a coating comprising one or more materials selected from the group consisting of ceramic materials, polymer-based materials, metallic materials, and mixtures thereof, in a process according to the present invention.

The present invention further relates to a device for performing the process according to the invention, said device comprising means for performing subtractive methods, and means for performing additive methods.

Said means for performing subtractive methods preferably consists of a five-axis grinding unit as is known to the skilled person, and which is already obtainable for a wide variety of applications. In order to achieve the required precision, said means for performing additive methods is actuated with the same degrees of freedom as the subtractive unit to apply the material also by using five axes.

In a preferred embodiment, the device has one or more material containers or cartridges, and holders for accommodating the containers or cartridges. In a particularly preferred embodiment, the device has at least two cartridges. The cartridges serve to receive the material from which the molded product according to the invention is formed. Therefore, an embodiment in which said at least two cartridges contain different materials is preferred. These different materials may have, for example, different colors or be materials with different material properties. In an alternatively preferred embodiment, said at least two cartridges contain the same material.

The device according to the invention preferably has means for attaching and positioning the cartridges. The means are preferably driven by a linear and/or rotatory motor. Such a design allows for an accurate and precise positioning of the cartridges.

Said means for attaching and positioning the cartridges is preferably a cartridge changer. Preferably, such cartridge changer and/or the cartridges have lightproof and/or airtight seals in order to avoid premature curing of the material contained in the cartridge.

In a particularly preferred embodiment, the cartridge changer has such a design that the cartridge that is being used for material application is brought into its application position by motor control. Preferably, this motor-controlled drive is also used to meter and apply the required amount of material.

1. A process for producing a molded product, comprising the following steps:
a) providing a blank;
b) machine-processing the blank using subtractive methods to obtain a framework structure;
c) machine-applying a coating to the framework structure obtained in step b) to obtain a raw molded product;
d) curing the coating;
e) machine-processing the coating using subtractive methods to obtain the desired molded product, wherein the application of the coatings, in particular, is effected using a device having an automated cartridge changer.

2. The process according to claim 1, wherein said process includes further steps for applying one or more further coatings, preferably by repeating steps c) to e).

3. The process according to claim 1, wherein said process is performed by an automated method.

4. The process according to claim 1, wherein said molded product is a dental restoration.

5. The process according to claim 1, wherein said blank comprises one or more materials selected from the group consisting of ceramic materials, polymer-based materials and metallic materials, as well as mixtures thereof.

6. The process according to claim 1, wherein said coating comprises one or more materials selected from the group consisting of ceramic materials, polymer-based materials and metallic materials, as well as mixtures thereof.

7. The process according to claim 1, wherein the material of the blank and the material of the coating is the same or different materials.

8. The process according to claim 1, wherein said subtractive method is selected from the group consisting of milling, grinding, laser ablation, and water jet cutting.

9. The process according to claim 1, wherein said application of the coating is effected by means of additive methods, preferably by extrusion, spraying, vapor deposition, deposition, infiltration, and/or immersion coating.

10. The process according to claim 1, wherein said curing of the coating is effected by polymerization, sintering, drying, cooling, pressure and/or irradiation.

11. A molded product obtainable by the process according to claim 1.

12. Use of a blank comprising a ceramic material, polymer-based material, and/or metallic material in a process according to claim 1.

13. Use of a coating material comprising a ceramic material, polymer-based material, and/or metallic material in a process according to claim 1.

14. A device for performing the process according to claim 1, said device comprising means for performing subtractive process steps, and means for performing additive process steps.
